(19) United States
(12) Patent Application Publication
(10) Pub. No.: US 2008/0125849 A1
(43) Pub. Date: May 29, 2008

(54) Delivery system catheter with rotating distal end

(76) Inventors: Janet Burpee, Fairhaven, NJ (US); Bradley Beach, Belmar, NJ (US); Rajesh Kalavalapally, Ocean, NJ (US); Andrew Filachek, Beachwood, NJ (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/983,828

(22) Filed: Nov. 13, 2007

Related U.S. Application Data
(60) Provisional application No. 60/858,543, filed on Nov. 13, 2006.

(51) Int. Cl. A61F 2/84

(52) U.S. Cl. 623/1.12

(57) ABSTRACT

A delivery catheter used for the delivery and deployment of self expanding stents. A distal end portion is made rotatable relative to a proximal end portion allowing unwinding of a self expanding stent around the longitudinal axis of the catheter. The rotation of the distal end portion allows for untwisting or partial untwisting of a self-expanding stent which has a torsional pre-load in a compressed state in the catheter.

### CROSS REFERENCE TO RELATED APPLICATION

This application claims the benefit of U.S. Provisional Patent Application No. 60/858,543, filed Nov. 13, 2006, the entirety of which is hereby incorporated by reference into this application.

### BACKGROUND OF THE INVENTION

1. Field of the Invention

The present invention relates generally to the method of delivery of expandable tubular structures capable of insertion into small spaces in living bodies and, more particularly, concerns a delivery system catheter which has a distal section or tip which can rotate around the catheter's longitudinal axis.

2. Description of the Related Art

A stent is a tubular structure that, in a radially compressed or crimped state, may be inserted into a confined space in a living body, such as an artery or other vessel. After insertion, the stent may be expanded radially to enlarge the space in which it is located. Stents are typically characterized as balloon-expanding (BX) or self-expanding (SX). A balloon-expanding stent requires a balloon, which is usually part of a delivery system, to expand the stent from within and to dilate the vessel. A self expanding stent is designed, through choice of material, geometry, or manufacturing techniques, to expand from the crimped state to an expanded state once it is released into the intended vessel. In certain situations the stent may twist as it is deployed from the delivery catheter. In this case, a delivery system catheter with a rotating tip would enable the stent to untwist or partially untwist before touching the vessel wall.

Stents are typically used in the treatment of vascular and non-vascular diseases. For instance, a crimped stent may be inserted into a clogged artery and then expanded to restore blood flow in the artery. Prior to release, the stent would typically be retained in its crimped state within a catheter and the like. Upon completion of the procedure, the stent is left inside the patient's artery in its expanded state. The health, and sometimes the life, of the patient depend upon the stent's ability to remain in its expanded state while not exerting significant or complex loads on the given vessel or duct (herein “vessel”).

Conventional delivery system catheters used in the delivery and deployment of self-expanding stents have a distal end which does not rotate significantly relative to the more proximal end. When deploying stents which exit the catheter with a stored energy, especially a stored torsional energy, the stent can twist relative to the catheter, and more importantly, relative to the centerline of the given vessel. If the stent continues to twist but does not completely untwist when it hits the vessel wall, there could be residual load, especially a torsional load, placed on the vessel. This load could potentially injure the vessel wall.

A similar problem could exist with respect to delivery of stent-like structures. An example of a stent-like structure would be a structure used with other components in a catheter-based valve delivery system. Such a stent-like structure holds a valve which is placed in a vessel. It is desirable to provide a catheter to allow a self expanding stent to unwind or partially unwind prior to deployment from the catheter.

### SUMMARY OF THE INVENTION

In accordance with the present invention, a catheter is constructed having a distal end portion that can rotate around its longitudinal axis at least about 45 degrees to more than about 360 degrees relative to a portion of the proximal end in order to allow a self expanding stent to unwind or partially unwind prior to or during deployment from the catheter. In one embodiment, the catheter is comprised of a pusher shaft, an outer sheath, optionally radiopaque markers, a guide wire lumen, appropriate valves and luer fittings as needed, and an end or distal portion capable of rotation about the longitudinal axis of the catheter and relative to the proximal end of the catheter. Alternatively, the end or distal portion of the catheter can be short such as about equal to the length of the crimped stent. The end or distal portion of the catheter can be long such as about nearly the entire length of the catheter. In this latter configuration most of the shaft of the catheter could rotate relative to the most proximal end including any valves or ports. In an alternate embodiment, the end or distal portion could also be any length in between the short and long lengths described above. The distal and proximal sections of the catheter which rotate relative to each other can be connected by for example, flexible sleeve, wires, threads, bearings, by one or more fibers (strings, threads, wires) of single or multiple filaments, or by any number of mechanisms that will allow the relative about 45 to more than about 360 degrees of rotation around the longitudinal axis.

### BRIEF DESCRIPTION OF THE DRAWINGS

The foregoing description, as well as further objects, features, and advantages of the present invention will be understood more completely from the following detailed description of presently preferred, but nonetheless illustrative embodiments in accordance with the present invention, with reference being had to the accompanying drawings, in which:

FIG. 1A is a schematic of a prior art self expanding stent delivery system which includes a section X-X.

FIG. 1B is a schematic diagram of detail Portion A shown in FIG. 1A.

FIG. 1C is a schematic diagram of detail Portion B shown in FIG. 1A.

FIG. 2A is a schematic diagram of an embodiment of self expanding stent delivery system in accordance with the present invention which includes a section X-X.

FIG. 2B is a schematic diagram of detail Portion A shown in FIG. 2A.

FIG. 3A is a schematic of a second embodiment of self expanding stent delivery system in accordance with the present invention which includes a section X-X.

FIG. 3B is a schematic diagram of detail Portion A shown in FIG. 3A.

FIG. 4A is a schematic of a third embodiment of self expanding stent delivery system in accordance with the present invention which includes a section X-X.

FIG. 4B is a schematic diagram of detail Portion A shown in FIG. 4A.

FIG. 5A is a schematic of a fourth embodiment of self expanding stent delivery system in accordance with the present invention which includes a section X-X.

FIG. 5B is a schematic diagram of detail Portion A shown in FIG. 5A.

FIG. 6A is a schematic of a fifth embodiment of self expanding stent delivery system in accordance with the present invention which includes a section X-X.

FIG. 6B is a schematic diagram of detail Portion A shown in FIG. 6A.

FIG. 7A is a schematic representation of a portion of a sixth embodiment of self expanding stent delivery system in accordance with the present invention.

FIG. 7B is a section view of FIG. 7A.

FIG. 7C is a schematic diagram of detail Portion A shown in FIG. 7B.

### DETAILED DESCRIPTION OF THE EMBODIMENTS

Reference will now be made in greater detail to the embodiments of the invention, examples of which are illustrated in the accompanying drawings. Wherever possible, the same reference numerals will be used throughout the drawings and the description to refer to the same or like parts.

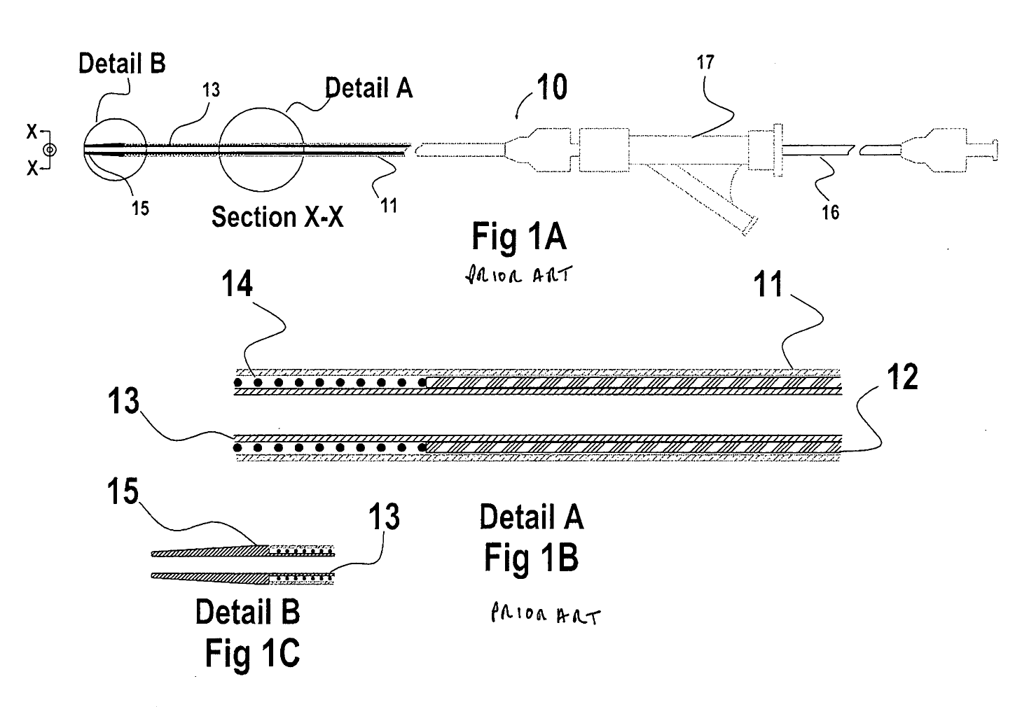

FIGS. 1A-1C are schematic diagrams of conventional self expanding stent delivery system 10 that is well known in the art. Delivery system 10 utilizes outer sheath 11 to hold stent 14 in a crimped (radially compact) state. Pusher assembly 12 positioned at the proximal end of stent 14 and reacts to the forces developed as outer sheath 11 is retracted during deployment of stent 14. Self expanding stent delivery system 10 typically has inner tube 13 (also known as a guide wire lumen) which goes over a guide wire (not shown), also used during a typical procedure. FIGS. 1A-1C also shows distal tip 15 used for delivery. Distal tip 15 is often integral to guide wire lumen 13. FIG. 1A also shows handle/hemostasis valve 17, which can be coupled to outer sheath 11 and pusher shaft 16 which is coupled to the pusher assembly 12. Delivery system 10 is designed such that outer sheath 11 and handle/hemostasis valve 17 can move longitudinally along the axis of outer sheath 11 relative to pusher assembly 12 and pusher shaft 16.

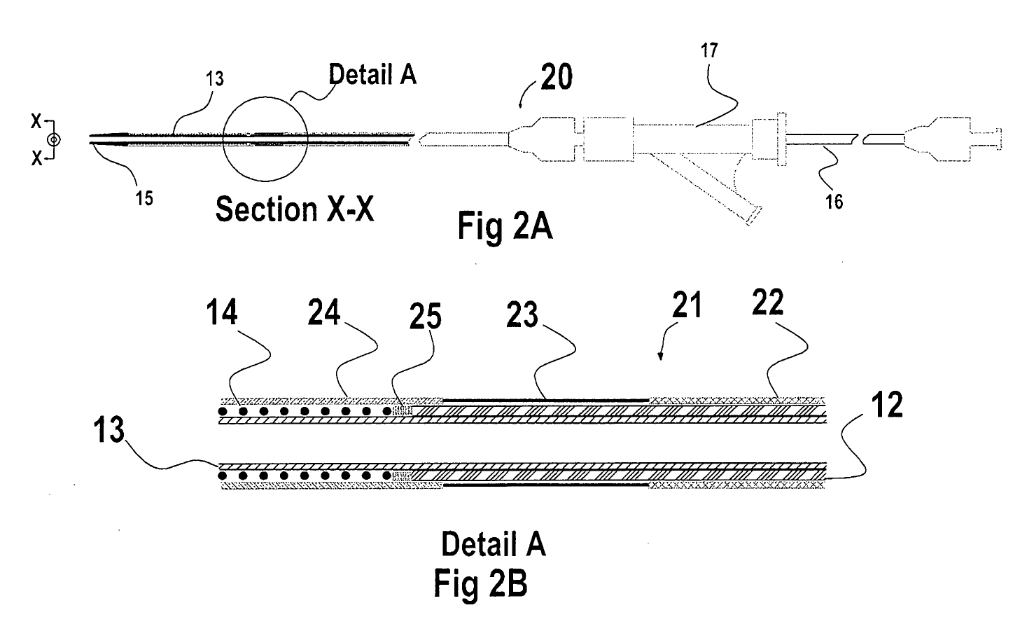

FIGS. 2A-2B are schematic diagrams of an embodiment of self expanding stent delivery system 20 in accordance with the present invention. This delivery system includes components of the conventional delivery system shown in FIGS. 1A-1C, which are labeled in FIGS. 2A-2B. Outer sheath 21 comprises distal portion 24, which is connected to proximal portion 22 by torsionally compliant element 23. Torsionally compliant element 23 allows distal portion 24 to rotate relative to proximal portion 22 and maintain sufficient axially rigidity such that all three elements can move longitudinally relative to pusher assembly 12 and pusher shaft 16. For example, torsionally compliant element 23 can be any one or combination of the following: a sleeve comprised of filaments, threads, fibers, or wires. The sleeve can overlap a portion of proximal portion 22 or distal portion 24 of outer sheath 21. A suitable sleeve is made of a flexible polymer or rubber. The individual fibers, threads, filaments or wires can overlap some of proximal portion 22 or distal portion 24 of outer sheath 21. Distal portion 24 can be short such as about equal to the length of the crimped stent 14. Alternatively, distal portion 24 can be long such as about nearly the entire length of outer sheath 21.

Delivery system 20 can include a type of thrust bearing element that allows rotation of the distal portion 24 relative to proximal portion 22 of outer sheath 21 and still transmits the required retraction force.

Proximal portion 22 of outer sheath 21 is coupled to handle/hemostasis valve 17 as in the conventional delivery system 10. Additionally, delivery system 20 includes thrust bearing element 25 between pusher assembly 12 and stent 14. Since stent 14 is in intimate contact with distal portion 24 of outer sheath 21. Thrust bearing element 25 can further allow stent 14 and distal portion 24 of outer sheath 21 to rotate relative to proximal portion 22 of outer sheath 21 and pusher assembly 12. For example, thrust bearing element 25 can be a conventionally defined thrust bearing element that reacts to loads parallel to the axis of rotation, but allows rotation along the axis.

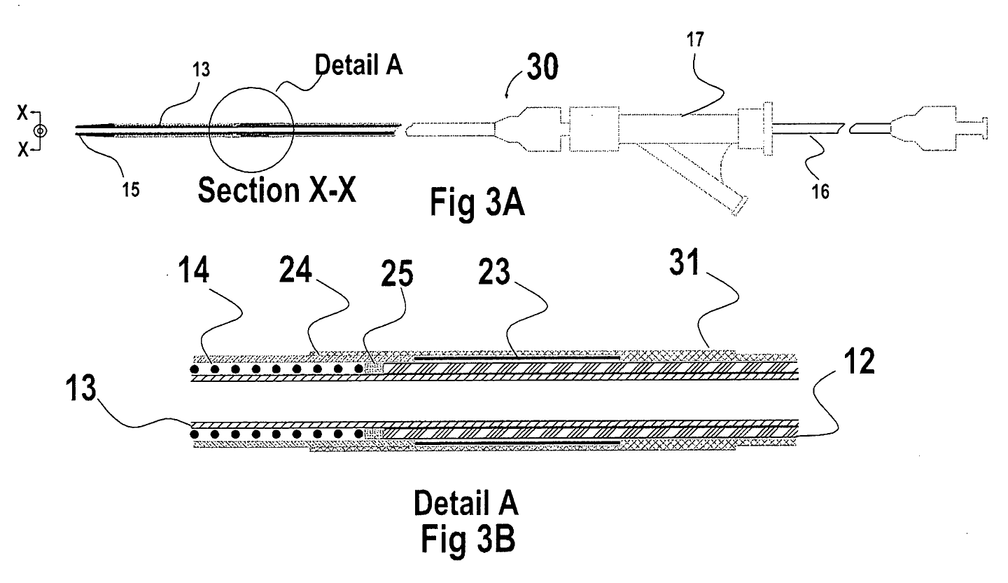

FIGS. 3A-3B are schematic diagrams of a second embodiment of self expanding delivery system 30 in accordance with the present invention. Delivery system 30 has all the same elements as delivery system 20 shown in FIGS. 2A-2B, which elements are labeled in FIGS. 3A-3B, except proximal portion 22 of outer sheath 21 is replaced by proximal outer sheath 31 that extends over torsionally compliant element 23 and part of distal portion 24 of outer sheath 21. Proximal outer sheath 31 can still rotate relative to the distal portion of the outer sheath, and by covering torsionally compliant element 23 can prohibit the leakage of bodily fluids into outer sheath 21.

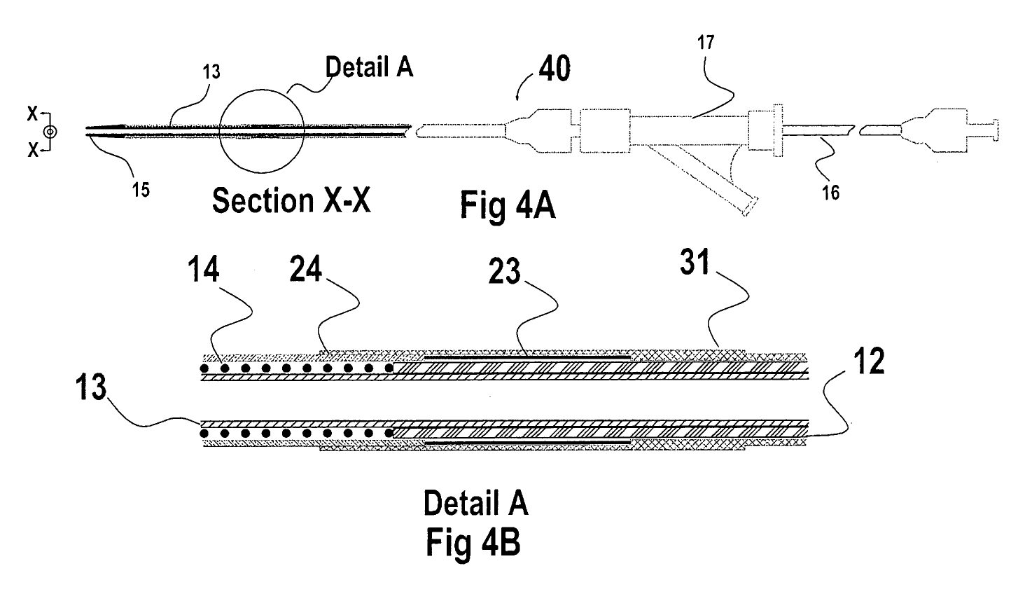

FIGS. 4A-4B are schematic diagrams of a third embodiment of self expanding delivery system 40 in accordance with the present invention. Delivery system 40 has all the same elements as delivery system 20 shown in FIGS. 2A-2B which are labeled in FIGS. 4A-4B, except thrust bearing element 25 between pusher assembly 12 and stent 14.

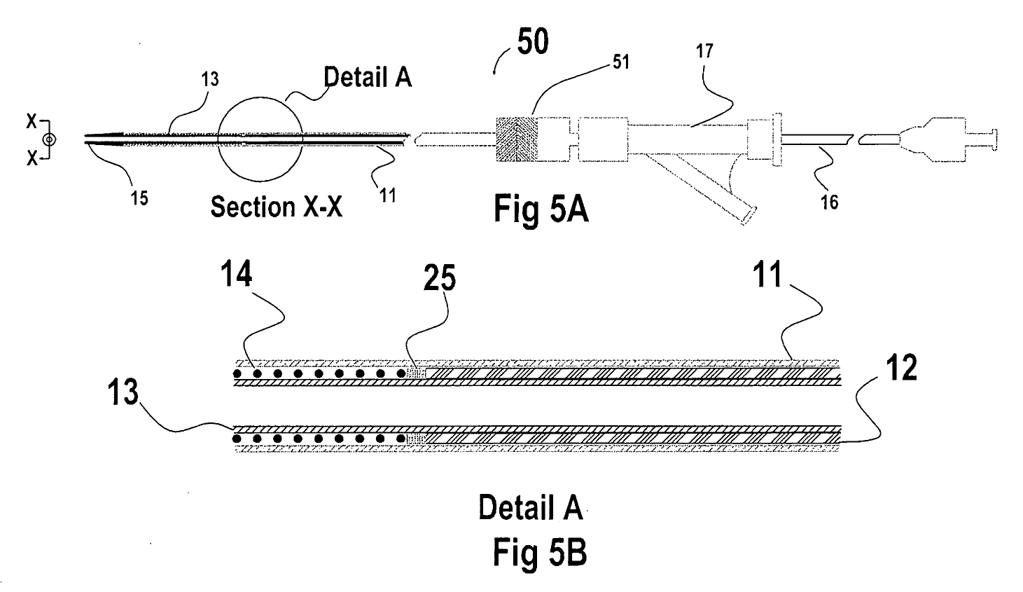

FIGS. 5A-5B are schematic diagrams of a fourth embodiment of self expanding delivery system 50 in accordance with the present invention. Delivery system 50 has all the same elements as conventional delivery system 10 as shown in FIGS. 1A-1C which elements are labeled in FIGS. 5A-5B, but also includes thrust bearing 51 coupling outer sheath 11 to handle/hemostasis valve 17, such that outer sheath 11 can rotate relative to pusher assembly 12 and pusher shaft 16 but still be longitudinally coupled to handle/hemostasis valve 17 to allow relative longitudinal movement of outer sheath 11 and handle/hemostasis valve 17 to pusher assembly 12 and pusher shaft 16. Delivery system 50 also includes thrust bearing element 25 between pusher assembly 12 and stent 14.

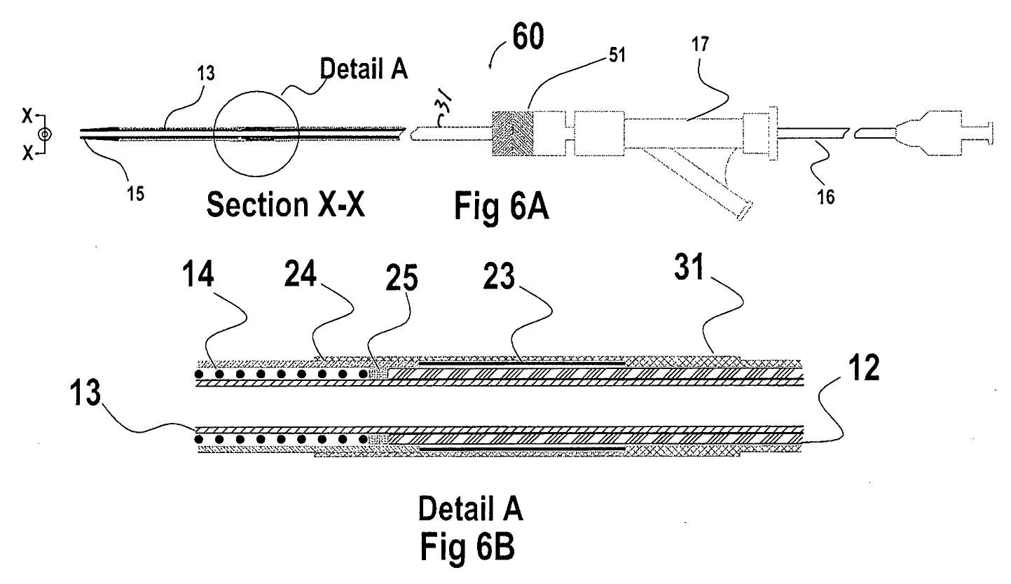

FIGS. 6A-6B are schematic diagrams of a fifth embodiment of self expanding delivery system 60 in accordance with the present invention. Delivery system 60 has all the same elements as delivery system 30 as shown in FIGS. 3A-3C which elements are labeled in FIGS. 6A-6B, but also includes thrust bearing 51 coupling outer sheath 31 to handle/hemostasis valve 17, such that proximal outer sheath 31 can rotate relative to pusher assembly 12 and pusher shaft 16 but still be longitudinally coupled to handle/hemostasis valve 17 to allow relative longitudinal movement of proximal outer sheath 31 and handle/hemostasis valve 17 to pusher assembly 12 and pusher shaft 16. The addition of thrust bearing 51 provides redundancy to delivery system 60.

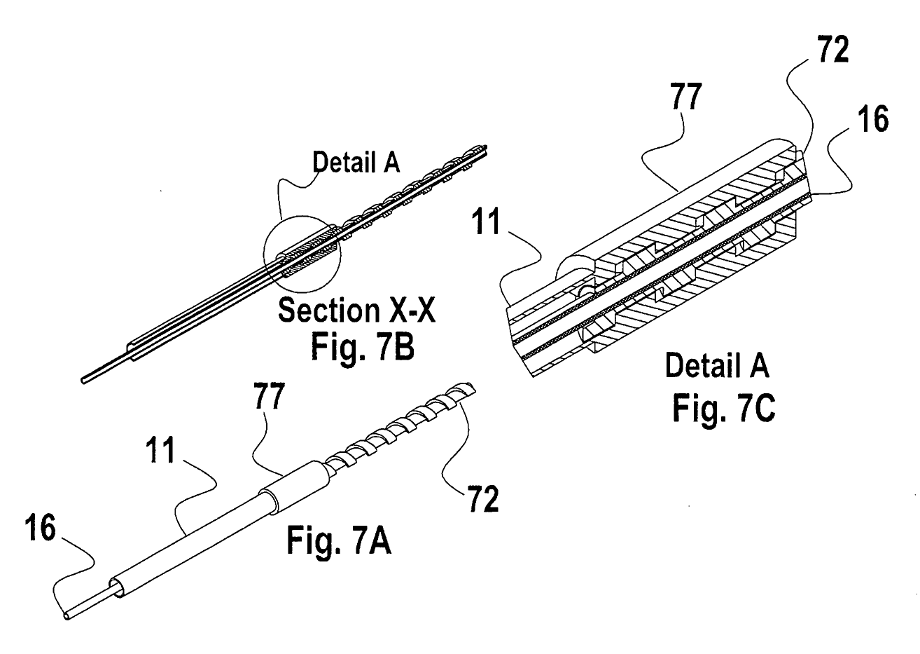

FIGS. 7A-7C are schematic representations of a portion of self expanding delivery system in an alternate embodiment in accordance with the present invention. FIGS. 7A-7C show an alternate version of the handle/hemostasis valve where the handle incorporates nut 77 which is coupled to outer sheath 11. Pusher assembly 12 incorporates lead screw 72 which is coupled to pusher shaft 16. In order to retract outer sheath 11, nut 77 rotates and traverses longitudinally along lead screw 72. The pitch and direction of lead screw 72 is such that as the stent is deployed, the stent unwinding is counteracted by the rotation of outer sheath 11 in accordance with the interaction of nut 77 and lead screw 72.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A stent delivery system comprising:
an outer sheath adapted to receive said stent in a crimped state, said outer sheath having a distal end portion coupled or integral with a proximal end portion;
wherein said distal end portion is capable of rotation about a longitudinal axis of said outer sheath relative to said proximal end portion prior to or during deployment of said stent.

2. The stent delivery system of claim 1 wherein said distal end portion can rotate at least about 45 degrees relative to said proximal end portion.

3. The stent delivery system of claim 1 wherein said distal end portion can rotate from about 45 degrees to more than about 360 degrees relative to said proximal end portion.

4. The stent delivery system of claim 1 wherein said distal end portion is coupled to said proximal end portion with a torsionally compliant element.

5. The stent delivery system of claim 5 wherein said torsionally compliant element is a flexible sleeve overlapping a portion of said distal end portion and a portion of said proximal end portion.

6. The stent delivery system of claim 6 wherein said flexible sleeve is formed of a flexible polymer or rubber.

7. The stent delivery system of claim 6 wherein said flexible sleeve comprises one or more of a filament, thread, fiber, or wire.

8. The stent delivery system of claim 1 further comprising:
a pusher assembly positioned at said proximal end portion; and
a thrust bearing element positioned between said pusher assembly and said stent.

9. The stent delivery system of claim 6 further comprising a proximal outer sheath covering said torsionally compliant element.

10. The stent delivery system of claim 1 further comprising:
a handle valve; and
a thrust bearing element coupling said handle valve to said proximal end portion of said outer sheath.

11. The stent delivery system of claim 1 wherein said distal end portion is coupled to said proximal end portion with a torsionally compliant element and a proximal outer sheath covers said torsionally compliant element, said system further comprising:
a handle valve; and
a thrust bearing element coupling said handle valve to an end portion of said proximal outer sheath.

12. The stent delivery system of claim 11 further comprising:
a pusher assembly positioned at said proximal end portion; and
a thrust bearing element positioned between said pusher assembly and said stent.

13. The stent delivery system of claim 1 further comprising:
a handle valve;
said handle valve having a nut, said nut being coupled to said outer sheath; and
a pusher assembly including a pusher shaft, said pusher assembly including a lead screw which is coupled to said pusher shaft,
wherein said outer sheath is retracted by said nut rotating and traversing longitudinally along said lead screw.

14. The stent delivery system of claim 1 wherein said distal end portion has a length of said stent.

15. The stent delivery system of claim 1 wherein said distal end portion has a length about the same as a length of said outer sheath.
